(19) United States
(12) Patent Application Publication
(10) Pub. No.: US 2008/0153894 A1
(43) Pub. Date: Jun. 26, 2008

(54) CYCLOOXYGENASE-2 INHIBITOR AND ANTIBACTERIAL AGENT COMBINATION FOR INTRAMAMMARY TREATMENT OF MASTITIS

(75) Inventors: Nancy J. Britten, Portage, MI (US); John W. Hallberg, Nashville, MI (US); Niki A. Waldron, Piscataway, NJ (US); Jeffrey L. Watts, Kalamazoo, MI (US)

Correspondence Address: PHARMACIA & UPJOHN, 7000 Portage Road, KZO-300-104, KALAMAZOO, MI, 49001, US

(73) Assignee: PHARMACIA CORPORATION, St. Louis, MO (US)

(21) Appl. No.: 12/037,556

(22) Filed: Feb. 26, 2008

Related U.S. Application Data
(63) Continuation-in-part of application No. 10/393,098, filed on Mar. 20, 2003.
(60) Provisional application No. 60/434,985, filed on Dec. 19, 2002.

(51) Int. Cl. A61K 31/415; A61P 31/04; A61P 25/00

(52) U.S. Cl. 514/406

(57) ABSTRACT

A method is provided for treatment of an infective condition in an udder of a milk producing animal. The method comprises intramammary administration of an antibacterial agent in combination therapy with a selective COX-2 inhibitor in therapeutically effective amounts of each. Also provided is a pharmaceutical composition comprising an antibacterial agent and a selective COX-2 inhibitor, together with one or more excipients, in a dosage form suitable for intramammary administration to a milk producing animal.

This application is a continuation-in-part of U.S. application Ser. No. 10/393,098, filed Mar. 20, 2003, now pending. This application also claims priority of U.S. Provisional Application Ser. No. 60/434,985 filed Dec. 19, 2002, now abandoned.

### FIELD OF THE INVENTION

The present invention relates to a method of treatment of an infective condition in an udder of a milk producing animal. The invention also relates to a pharmaceutical composition suitable for intramammary administration for treatment of an infective condition in an udder, and to a process for preparing such a composition.

### BACKGROUND OF THE INVENTION

Mastitis is an inflammation of the mammary gland of milk producing animals, for example dairy cows, most often caused by bacterial infection. Bacteria enter through the teat canal of the animal and can cause acute, clinical or sub-clinical mastitis. Over 135 organisms have been documented as causative pathogens for bovine mastitis. Three of the major groups of pathogens are gram-positive cocci, gram-negative bacilli and gram-positive bacilli. Hygiene, environmental factors and metabolic disturbances deriving from high milk yield combine to create conditions favorable to onset of mastitis. An increased somatic cell count, associated with mastitis, is positively correlated with infection and negatively correlated with milk production. Frequently, an infected cow must be removed from the herd and dried up. Mastitis often affects a cow during its entire life unless the disease is properly treated Infection rates average from 10% to 30% of the cows in a typical herd, with losses per cow ranging from $185 to $250 per cow per year. Bovine mastitis is the most economically costly disease to the dairy industry, with losses estimated at two billion dollars annually in the United States alone. The majority of these losses are due to reduced milk production.

Very few antibacterial agents possess anti-inflammatory, antipyretic or analgesic properties in addition to their antibacterial activity. Therefore, treating an infective condition with an antibacterial agent alone typically does not alleviate the inflammation, pain, swelling, fever and other complications that often accompany such an infective condition. These problems are usually not totally resolved until the causal organism of the infective condition has been eliminated or reduced to a subpathogenic population by the antibacterial agent.

Treatment of an infective condition having an inflammatory component with an anti-inflammatory agent alone can reduce inflammation, swelling, pain, fever and other complications, but does not treat the underlying infective condition.

The use of antibacterial agents and the use of anti-inflammatory agents for treatment of mastitis in milk producing animals are well known.

International Patent Publication No. WO 99/20259 discloses a combination of thiamphenicol and diclofenac for use in veterinary medicine to treat infections with associated inflammatory conditions.

U.S. Pat. No. 5,756,529 to Isakson & Talley discloses a method of using pyrazolyl benzenesulfonamide compounds to treat inflammation in a companion animal. Such compounds are said to be useful for treatment of pain, fever, joint disease, traumatic injury, arthritis, myositis, tendinitis, equine colic, mastitis, peritonitis, skin conditions, burns, gingivitis, hypersensitivity, conjunctivitis, eye inflammation, swelling and myocardial ischemia.

International Patent Publication No. WO 02/06865 discloses a composition comprising one or more bioactive substances in a non-aqueous carrier wherein the composition has been adjusted to have a water activity of about 0.2 to about 0.5. Parenteral, topical, oral, intravaginal, rectal and intramammary administration is proposed. Among the bioactive agents listed are anti-infectives, antineoplastics, immunomodulators, antipyretics, analgesics and anti-inflammatory agents (e.g., cyclooxygenase-2 (COX-2) inhibitors).

International Patent Publication No. WO 01/60409 discloses a paste composition comprising a therapeutic agent, fumed silica, a viscosity modifier and a hydrophilic carrier; wherein the therapeutic agent is selected from insecticides, acaricides, parasiticides, antibiotics, growth enhancers, oil-soluble NSAIDs, avermectins, milbemycins, nordulisporic acid, estrogens, progestins, phenylpyrazoles, substituted pyridyl methyl derivatives and COX-2 inhibitors. Oral, topical, dermal and subdermal routes of administration are contemplated for the paste composition. Such compositions are said to have application in veterinary practice in treatment of diseases such as pneumonia, mastitis, metritis, rhinitis and bronchitis.

U.S. Patent Application Publication No. 2002/0032228 discloses use of a heterocycle containing compound, for example a diphenyl heterocycle derivative, to treat diarrheal diseases, whooping cough, anthrax, smooth muscle contraction conditions and mastitis. Celecoxib and rofecoxib are listed as preferred diphenyl heterocycle derivatives.

All of the above patents and articles are incorporated herein by reference but are not necessarily prior art under patent statutes.

The use of anti-inflammatory agents such as corticosteroids and non-selective NSAIDs can cause serious side effects. Non-selective NSAIDs can produce side effects that include gastrointestinal toxicity, gastrointestinal irritation, upper gastrointestinal ulceration and bleeding, life threatening ulcers, renal toxicity, blockage of platelet aggregation, and hepatic damage. Side effects associated with corticosteroid use include hypertension, arteriosclerosis, diabetes, hyperglycemia, osteoporosis, electrolyte imbalance, slow healing of infections, elevated blood cholesterol, detrimental effects on the functioning of both cellular and humoral defense mechanisms, pituitary-adrenal suppression, fluid and salt retention that can aggravate heart or kidney disease, and increased incidences of cataracts and glaucoma.

Although the references cited above disclose a number of anti-mastitis compositions, there still exists a need in the art for methods of treatment and for pharmaceutical compositions having one or more of the following advantages over prior art methods and/or compositions: (a) targeted delivery of an antibacterial agent and a safe, effective anti-inflammatory agent to the site of an udder infection, (b) improvement of the therapeutic index of an active agent while decreasing its general toxicity and minimizing the risk of systemic effects, (c) effective treatment for the inflammatory component as well as the infectious component of mastitis, (d) effective treatment of the pain, inflammation, fever, swelling, infection and complications associated with mastitis, (e) decreased time required to alleviate an infective condition having an inflammatory component, (f) reduction of side effects, (g) efficacy against a wide variety of infectious organisms, (h) minimal to no irritation after administration of the composition, (i) potential to administer a lower dose of a therapeutic agent while still providing efficacy, and (j) potential to administer a higher dose of an antibacterial agent without increased side effects.

### SUMMARY OF THE INVENTION

Novel methods of treatment and pharmaceutical compositions having some or all of the advantageous attributes described above have now been developed. In particular, there is provided a novel method of treatment of an infective condition in an udder of a milk producing animal. The method comprises intramammary administration to the animal of an antibacterial agent in combination therapy with a selective COX-2 inhibitor in therapeutically effective amounts of each.

Preferably at least one, more preferably both, of the antibacterial agent and the selective COX-2 inhibitor are administered by intramammary infusion.

The antibacterial agent and the selective COX-2 inhibitor can be administered sequentially in either order or simultaneously. In one embodiment the antibacterial agent and the selective COX-2 inhibitor are administered as a single pharmaceutical composition comprising, in addition to the antibacterial agent and the selective COX-2 inhibitor, a vehicle that comprises at least one pharmaceutically acceptable excipient.

The method is useful for treatment of mastitis and other diseases of the udder in a milk producing animal, and is efficacious in a wide variety of infective disorders involving a wide variety of infectious organisms.

When administered by the intramammary route, the combination therapy of the invention provides enhanced treatment options as compared to intramammary administration of either the selective COX-2 inhibitor or the antibacterial agent alone, and as compared to administration of the COX-2 inhibitor and/or the antibacterial agent by routes other than intramammary.

Combination therapy according to the invention provides effective treatment for both the infectious and the inflammatory components of an infective conditions and can reduce the time required to resolve the infective condition and the associated inflammation.

The term “treatment” herein includes administration of a therapeutic agent to a non-lactating animal, for example a dry cow, which does not yet show clinical signs of mastitis, but which is at risk for developing clinical mastitis. The invention therefore provides a method for reducing risk of developing clinical mastitis in a future lactating animal at such risk, the method comprising intramammary administration to the animal of an antibacterial agent in combination therapy with a selective COX-2 inhibitor, in therapeutically effective amounts of each.

In a preferred embodiment, however, combination therapy according to the invention is administered to a milk producing animal that has clinical signs of mastitis The invention therefore provides a method for treating clinical mastitis in a milk producing animal, the method comprising intramammary administration to the animal, of an antibacterial agent in combination therapy with a selective COX-2 inhibitor, in therapeutically effective amounts of each.

Certain antibacterial agents, while being very effective against infective bacteria, are associated with a risk of undesirable side effects, such as transient redness, swelling and inflammation of the udder. Acceptable dosages of some antibacterial agents can be practically limited by the need to minimize risk of such side effects. The combination therapy method of the present invention minimizes these risks, thereby providing improved treatment of mastitis and other diseases of the udder.

It is believed, without being bound by theory, that certain antibacterial agents, when administered to certain animals, can promote release of endotoxins that in turn sets off a TNF (tumor necrosis factor alpha) mediated response, and it is further believed that such response can be blocked or mitigated by the selective COX-2 inhibitor.

While conventional NSAIDs inhibit both isoforms of the cyclooxygenase (COX) enzyme, selective COX-2 inhibitors target COX-2 with minimal to no effect on COX-1, thus effectively reducing inflammation while producing fewer and less severe side effects than those that can occur as a result of treatment with non-selective NSAIDs or corticosteroids.

Combination therapy as described herein therefore provides safes effective treatment for both the inflammatory component as well as the infectious component of mastitis and other diseases of the udder. Such combination therapy can also reduce or alleviate pain, swelling and fever associated with an infection of the udder. Further, the intramammary administration route according to the present method provides targeted delivery of the antibacterial agent and the selective COX-2 inhibitor to the site of infection and/or inflammation in the udder.

In a further embodiment combination therapy according to the invention can improve the therapeutic index of an active agent by decreasing its general toxicity and minimizing the risk of systemic side events. Therapeutic index is a measure of the margin between a therapeutically effective dose and a toxic dose of a drug and is typically expressed as the ratio of LD₅₀ (a dose lethal to 50% of a population) to ED₅₀ (a dose therapeutically effective in 50% of the population).

Combination therapy according to the invention can also allow the administration of a lower dose of a therapeutic agent while still providing efficacy.

In another embodiment the invention provides a pharmaceutical composition comprising an antibacterial agent and a selective COX-2 inhibitor, together with one or more excipients, in a dosage form suitable for intramammary administration to a milk producing animal. Such a composition can provide effective treatment for the inflammatory component as well as the infectious component of mastitis and other diseases of the udder. Preferably the antibacterial agent and the selective COX-2 inhibitor are dissolved or dispersed in a liquid vehicle comprising one or more excipients and the composition is adapted for intramammary infusion.

In accordance with this embodiment, a method is provided for effecting targeted delivery of an antibacterial agent and a selective COX-2 inhibitor to a site of infection in an udder of a milk producing animal, the method comprising intramammary administration to the animal of a composition as described above.

Compositions of the invention can provide effective treatment for pain, inflammation, fever, swelling, infection and other complications associated with mastitis. A further benefit of preferred compositions is that they produce minimal to no irritation after administration.

Preferred methods and compositions can have additional advantages. For example, a preferred method enables suitably short milkout times. Milkout time for a lactating cow is the period of time from administration of a mastitis treatment to resumption of production of saleable milk. Following such administration, the concentration of active agent(s) in milk must fall to a level acceptable to the appropriate regulatory body before the milk is deemed suitable for human consumption. A suitably short milkout time reduces monetary losses to a dairy farmer caused by a mastitis outbreak.

Alternatively or in addition, a preferred method enables a low milk withholding time post calving after dry cow mastitis treatment, with no active agent residues in the offspring.

Alternatively or in addition, a preferred method enables a zero day slaughter meat withdrawal period following mastitis treatment. This attribute is especially important since it allows a farmer to dispose of a treated cow at any time it is financially advantageous to do so, rather than being required to keep and feed a cow for a specified amount of time after its treatment.

The present invention thus provides solutions to several long standing problems in the art and possesses one or more advantages over methods and compositions of prior art. Other features, advantages and benefits of the invention will be apparent from the description that follows.

### DETAILED DESCRIPTION OF THE INVENTION

The invention provides a method of treatment of an infective condition in an udder of a milk producing animal. The method comprises intramammary administration of an antibacterial agent in combination therapy with a selective COX-2 inhibitor in therapeutically effective amounts of each as defined herein.

An “infective condition” herein includes any disease, disorder or condition mediated by a pathogenic bacterium or that is otherwise responsive to treatment with an antibacterial agent such as an antibiotic drug, whether or not accompanied by pain, fever or inflammation. The invention is, however, especially drawn to such conditions having a component of pain, fever or inflammation.

The term “intramammary administration” herein encompasses any means of administration into the udder via a teat canal. Examples of intramammary administration means include, but are not limited to, infusion, injection, insertion, implantation, propulsion and the like. A preferred means is infusion as defined hereinbelow.

A “milk producing animal” can be a female of any mammalian species but is preferably an animal raised for the purpose of providing milk, e.g., a cow, a goat or a sheep, and encompasses such animals whether or not they are lactating at the time of the infective condition or at the time of treatment.

It will be understood that reference herein to methods involving and compositions comprising “an antibacterial agent” embraces such methods and compositions wherein more than one antibacterial agent is used. Further, more than one selective COX-2 inhibitor can optionally be used.

The term “antibacterially effective amount” as used herein refers to an amount of an antibacterial agent that is sufficient, when administered by the method of the invention, to reduce, relieve, prevent or delay onset of one or more symptoms of an infective condition being treated, or to reduce numbers and/or activity of a causal organism.

The term “anti-inflammatorily effective amount” as used herein refers to an amount of an anti-inflammatory agent, in this case a selective COX-2 inhibitor, that is sufficient, when administered by the method of the invention, to reduce, relieve, prevent or delay onset of one or more symptoms of an inflammatory condition being treated.

A selective COX-2 inhibitor is a compound that selectively inhibits cyclooxygenase-2 (COX-2) activity. The terms “selective COX-2 inhibitor” and “selective cyclooxygenase-2 inhibitor” interchangeably refer to a therapeutic compound that selectively inhibits the COX-2 isoform of the enzyme cyclooxygenase, with less significant inhibition of cyclooxygenase-1 (COX-1). As used herein the term “selective COX-2 inhibitor” also refers to a prodrug or salt that is converted in vivo to a compound that exhibits selective inhibition of COX-2 relative to COX-1. Preferred selective COX-2 inhibitors exhibit a selectivity factor of at least about 10, more preferably at least about 50 and still more preferably at least about 100, wherein “selectivity factor” is defined as IC₅₀(COX-1)/IC₅₀(COX-2), IC₅₀ being the concentration of a compound producing 50% inhibition of enzyme activity in an in vitro or in vivo test.

The term “combination therapy” herein means a treatment regimen wherein the antibacterial agent and the selective COX-2 inhibitor are administered individually or together in such a way as to provide a beneficial effect from co-action of these therapeutic agents. Such beneficial effect can include, but is not limited to, pharmacokinetic or pharmacodynamic co-action of the therapeutic agents. Combination therapy can, for example, enable administration of a lower dose of one or both agents than would normally be administered during monotherapy, thus decreasing risk or incidence of adverse effects that may be associated with higher doses in certain animals. Alternatively, combination therapy can result in increased therapeutic effect at the normal dose of each agent in monotherapy. Alternatively, combination therapy can maximize therapeutic effect at higher doses. “Combination therapy” herein is not intended to encompass administration of two or more therapeutic agents as part of separate monotherapy regimens that incidentally and arbitrarily result in sequential or simultaneous treatment.

The term “therapeutically effective amount” as used herein refers to an amount of a compound being administered that is sufficient to reduce, relieve, prevent or delay onset of one or more symptoms of a condition being treated. The phrase “an therapeutically effective amounts of each” means that when administered in combination therapy according to the method of the invention, the amount of the antibacterial agent and the amount of the selective COX-2 inhibitor are sufficient to provide both an antibacterial effect and an anti-inflammatory effect. Such amounts can be the same as, greater or less than the amount of antibacterial agent or the amount of selective COX-2 inhibitor that are therapeutically effective when used in monotherapy.

Administration of the antibacterial agent and the selective COX-2 inhibitor typically is carried out over a defined time period (usually minutes, hours, days or weeks depending upon the combination selected). These therapeutic agents can be administered in a sequential manner, that is, at different times, typically separated by no more than about 24 hours, or in a substantially simultaneous manner.

When administered simultaneously, the antibacterial agent and the selective COX-2 inhibitor can be administered in separate dosage forms or in coformulation, i.e., in a single dosage form. When administered in separate dosage forms, the antibacterial agent is administered as a pharmaceutical composition comprising said antibacterial agent, typically dispersed in a first pharmaceutically acceptable vehicle, and the selective COX-2 inhibitor is administered as a separate pharmaceutical composition comprising said selective COX-2 inhibitor, typically dispersed in a second pharmaceutically acceptable vehicle that can be similar to or different from the first vehicle. In a preferred embodiment, both agents are co-dispersed in the same vehicle and administered in a single operation, most preferably by intramammary infusion.

The term “dispersed” in the present context means dissolved (i.e., molecularly dispersed) or colloidally dispersed, for example as an emulsion, suspension or solid dispersion. Typically at least one of the therapeutic agents is suspended in solid particulate form in a liquid vehicle.

A pharmaceutically acceptable carrier or vehicle is one that has no unacceptably injurious or toxic effect on the animal when administered as a component of a composition by intramammary administration in an amount required herein. No excipient ingredient of such a carrier or vehicle reacts in a deleterious manner with another excipient or with the therapeutic agent(s) in a composition.

“Intramammary infusion” is an operation wherein a liquid composition is caused to flow into an udder via a teat canal, regardless of the timescale involved in the present context, “infusion” and “injection” are substantially synonymous. For example, a liquid composition can be administered by inserting the cannula nozzle of a mastitis syringe into the external orifice of the teat canal and injecting the composition through the nozzle into the udder.

The terms “Labrafil™ M-1944CS,” “Labrafil™ M-1966CS,” and “Labrafil™ WL-2609BS” refer to oils comprising polyglycolized glycerides prepared by an alcoholosis reaction of natural triglycerides with polyethylene glycols.

A composition useful in the method of the invention can be prepared in a conventional manner and comprises one or more excipients or auxiliaries that for example, facilitate processing of the active agent(s) into preparations that can be used pharmaceutically. A pharmaceutically active agent can be present in the composition as drug particles, powders, granules, nanoparticles, microparticulates, microspheres, in lyophilized form, in dissolved form or the like. Compositions suitable for intramammary infusion are liquid and include, but are not limited to, solutions, suspensions, slurries, emulsions, reconstituted compositions and the like. A suitable liquid composition can be aqueous based or non-aqueous based, and can comprise one or more excipients selected from stabilizers, thickening agents, suspending agents, dispersing agents, solubilization agents, antioxidants, preservatives, isotonic agents, buffering agents, surfactants, other conventional pharmaceutical additives and the like. A suspension useful according to invention can be prepared by adding appropriate excipients to a liquid vehicle and mixing to form a pharmaceutically acceptable vehicle. Next an antibacterial agent and/or a selective COX-2 inhibitor are added to the vehicle and mixed to form a uniform suspension.

Other suitable dosage forms for intramammary administration include, but are not limited to, suppositories, conventional or in situ forming implants, conventional or in situ forming gels, ointments, aerosol sprays, nebulized solutions and the like.

An intramammary suppository composition useful according to the invention can be prepared by mixing at an elevated temperature, until uniformly distributed, an antibacterial agent and/or a selective COX-2 inhibitor with a non-irritating pharmaceutically acceptable carrier that is solid at room temperature but liquid at body temperature (such as cocoa butter, beeswax, synthetic mono-, di- or triglycerides, fatty acids, polyethylene glycols and the like), to form a suppository.

Methods for the preparation of in situ forming gels applicable for use with the invention are substantially described in the literature, for example in the patents individually cited below and incorporated herein by reference.

U.S. Pat. No. 4,861,760 to Mazuel & Friteyre.

U.S. Pat. No. 5,192,535 to Davis et al.

U.S. Pat. No. 5,587,175 to Viegas et al.

European Patent No. 0 424 043.

An aerosol spray for intramammary administration can be in the form of a solution, dry powder or cream. The aerosol spray can use, for example, a pressurized pack or nebulizer and a suitable propellant.

In another embodiment the active agent can be delivered using a sustained release system. Various sustained release materials have been established and are well known by those skilled in the art.

Optionally, administration of the therapeutic agents described above can take place in further combination with other biologically active agents and non-drug therapies.

In all embodiments of the invention an antibacterial agent and a selective COX-2 inhibitor are administered locally to the udder of a milk producing animal. An essential requirement for successful mastitis therapy is that an antibacterial agent must reach the site of infection at a concentration near or higher than the minimal inhibitory concentration and that such concentration must be maintained for a certain minimal time. There are significant differences among antibacterial agents in their ability to reach an infected site in the udder, and these are greater than the differences in their intrinsic antibacterial activities. One advantage of local administration according to the invention is that the antibacterial agent and the selective COX-2 inhibitor are preferentially directed towards their site of action, resulting in more rapid onset of therapeutic action and more complete delivery to the site of infection, compared with other routes of administration such as intramuscular, subcutaneous and oral routes. Local administration can allow the total therapeutic dose for a given effect to be decreased and avoids the hepatic first pass effect. In addition, local administration decreases or eliminates secondary effects, especially those linked to one or both of the active agents, at sites other than the site of infection. Local administration of an active agent can also improve its therapeutic index by decreasing its general toxicity and minimizing risk of undesirable systemic effects. Therapeutic index is a measure of the margin between a therapeutically effective dose and a toxic dose of a drug and is typically expressed as the ratio of LD₅₀ (a dose lethal to 50% of a population) to ED₅₀ (a dose therapeutically effective in 50% of the population).

The invention provides, in a further embodiment, a pharmaceutical composition adapted for intramammary administration, comprising an antibacterial agent in an antibacterially effective amount and a selective COX-2 inhibitor in an anti-inflammatorily effective amount, together with one or more excipients. Such a composition is suitable for single administration providing combination therapy in accordance with the method of the invention.

A preferred composition is one that is adapted for intramammary infusion, and comprises a liquid vehicle comprising one or more excipients and having dispersed therein an antibacterial agent in an antibacterially effective amount and a selective COX-2 inhibitor in an anti-inflammatorily effective amount

Antibacterial agents applicable for use according to the invention include any such agents that are effective for treatment and/or prevention of a mammary disorder and/or complications associated therewith. Suitable antibacterial agents include, but are not limited to, beta-lactam antibacterials such as natural and synthetic penicillin type agents including penam penicillins (such as benzyl penicillin, phenoxymethyl penicillin, coxacillin, nafcillin, methicillin, oxacillin, amoxycillin, temocillin, ticarcillin and the like), penicillinase-stable penicillins, acylamino and carboxypenicillins (such as piperacillin, azlocillin, meziocillin, carbenicillin, temocillin, ticarcillin and the like), and broader spectrum penicillins (such as streptomycin, neomycin, framycetin, gentamicin, apramycin, amikacin, spectinomycin, amoxycillin, ampicillin and the like), cephalosporins, macrolides (such as tylosin, tilmicosin, aivlosin, erythromycin, azithromycin, spiramycin, josamycin, kitasamycin and the like), lincosamides (such as lincomycin, clindamycin, pirlimycin and the like), pleuromutilins (such as tiamulin, valnemulin and the like), polypeptides, glycopeptides (such as vancomycin and the like), polymixins (such as polymixin B, polymixin E and the like), sulfonamides (such as sulfamethazine, sulfadiazine, sulfatroxazole, sulfamethoxypyridazine, sulfanilamide, sulfamethoxazole, sulfisoxazole, sulfamethizole, silver sulfadiazine, mafenide and the like, alone or in combination with trimethoprim), chloramphenicol, thiamphenicol, florfenicol, tetracycline type agents (such as tetracycline, chlortetracycline, oxytetracycline, domeclocycline, doxycycline, minocycline and the like), quinolones and fluoroquinolones (such as ciprofloxacin, enoxacin, grepafloxacin, levoffoxacin, lomeffoxacin, norfloxacin, ofloxacin, sparfloxacin, trovafloxacin, cinocacin, nalidixic acid and the like), tiamulin, colistin, meropenem, sulbactam, tazobactam, methacycline, pyrimethamine, sulfacetamide, oxazolidinones, e.g., eperezolid, linezolid, N-((5S)-3-(3-fluoro-4-(4-(2-fluoroethyl)-3-oxy-1-piperazinyl)phenyl-2-oxy-5-oxazolidinyl)methyl)acetamide, (S)-N-((3-(5-(3-pyridyl)thiophen-2-yl)2-oxy-5-oxazolidinyl)methyl)acetamide, (S)-N-((3-(5-(4-pyridyl)pyrid-2-yl)-2-oxy-5-oxazolidinyl)methyl)acetamide hydrochloride, 2,2-difluoro-N-({(5S)-3-[3-fluoro-4-(4-glycoloylpiperazin-1-yl)phenyl]-2-oxo-1,3-oxazolidin-5-yl)methyl)ethanethioamide and the like, aminoglycosides (kanamycin, tobramycin, netilmicin and the like), aminocyclitols, amphenicol, ansamycin, carbaphenem, cephamycin, rifampicin, monobactam, oxacephem, streptogramins (such as quinupristin, dalfopristin and the like), cycloserines, mupirocin, urea hydroxamates, folic acid analogs (such as trimethoprim and the like), antibiotic-type antineoplastic agents (such as aclarubicin, actinomycin D, actinoplanone, aeroplysinin derivative, Nippon Soda anisomycins, anthracycline, azino-micyin-A, busucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-A1b, fostriecin, glidobactin, gregatin-A, grincamycin, herbimycin, idarubicin, illudins, kazusamycin, kesarirhodins, menogaril, mitomycin, mitoxantorone, mutamycin, mycophenolate mofetil, neoenactin, oxalysine, oxaunomycin, peplomycin, pilatin, pirarubicin, porothramycin, pyrindamycin A, rapamycin, rhizoxin, rodorubicin, sibanomicin, siwenmycin, sorangicin-A, sparsomycin, steffimycin B, talisomycin, terpentecin, thrazine, tricrozarin A, zorubicin and the like), systemic antibacterials (such as 2,4-diaminopyrimidine), nitrofuran sulfones, marbofloxacin and the like, and combinations thereof.

It should be understood that any reference herein to a particular drug compound includes tautomers, stereoisomers, enantiomers, salts, hydrates and prodrugs of that compound and is not specific to any one solid state form of the drug.

Preferred antibacterial agents applicable for use according to the invention are cephalosporins including, but not limited to, ceftiofur hydrochloride, ceftiofur free acid, e.g., ceftiofur crystalline free acid, ceftiofur sodium, other ceftiofur salts, cephalexin, cephradine, cefquinome, cephacetrile, cephalonium, cefuroxime, cefazidime, cefoperazone, sodium cephemethcarboxylate, cephem heptahydrate, cephalosporin di- or tri-hydrate, cephadroxil monohydrate, cephazolin sodium monohydrate, cefiximine, ceftaxime, ceftizoxime, ceftriaxone, o-formylcefamandole, salts of 3-acetoxymethyl-7-(iminocetamido)-cephalosporanic acid derivatives, monohydrate of 7-(D-alpha-amino-alpha-(p-hydroxyphenyl)acetamino)-3-methyl-3-cephem-1-carboxylic acid, hydrochloride salt of syn-7-((2-amino-1-thiazolyl)(methoxyimino)acetyl)amino)-3-methyl-3-cephem-4-carboxylic acid, cephem acid addition salts, (pivaloyloxy)methyl 7-beta-(2-(2-amino-4-thiazolyl)acetamido)-3-(((1-(2-(dimethylamino)ethyl)-1H-tetraazol-5-yl)thio)methyl)-3-cephem-4-carboxylate, cephalexin, cephalexin monohydrate, 7-(D-2-naphthyglycylamino)-3-methyl-3-cephem-4-carboxylic acid tetrahydrate and the like. The most preferred cephalosporins for use according to the present invention are ceftiofur and pharmaceutically acceptable salts thereof. Especially preferred are ceftiofur free acid, most especially in crystalline form, and ceftiofur hydrochloride.

Where the antibacterial substance is ceftiofur or a salt thereof, a preferred concentration range in a composition of the invention for intramammary infusion is about 1 to about 1000 mg/ml, more preferably about 5 to about 750 mg/ml, and still more preferably about 10 to about 100 mg/ml. For antibacterial substances other than ceftiofur, suitable concentration ranges that are antibacterially equivalent can be determined by one of skill in the art based upon published data.

Examples of selective COX-2 inhibitors applicable for use according to the invention include, but are not limited to, the compounds described below and include tautomers, stereoisomers, enantiomers, salts, hydrates, prodrugs and combinations thereof. Any such selective COX-2 inhibitory drug or prodrug known in the art can be used.

A preferred selective COX-2 inhibitory drug useful herein is a compound of formula (I):

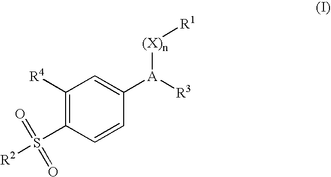

or a prodrug or pharmaceutically acceptable salt thereof, wherein:
A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;
X is O, S or CH₂;
n is 0 or 1;
R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is methyl, amino or aminocarbonylalkyl;
R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R⁴ is selected from hydrido and halo.

A particularly preferred group of selective COX-2 inhibitory drugs are compounds having the formula (II):

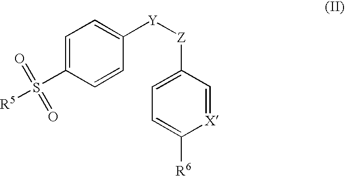

where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Another particularly preferred group of selective COX-2 inhibitory drugs are compounds having the formula (III):

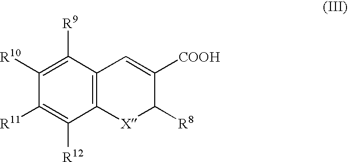

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy or aryl; and pharmaceutically acceptable salts thereof.

A particularly useful compound of formula (III) is (S)-6,8-dichloro-2-(trifluoromethyl)-2H-1-benzopyran-3-carboxylic acid.

Another particularly preferred group of selective COX-2 inhibitory drugs are 5-alkyl-2-arylaminophenylacetic acids and derivatives thereof. Particularly useful compounds of this class are lumiracoxib and pharmaceutically acceptable salts thereof.

Illustratively, celecoxib, deracoxib, valdecoxib, parecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide, and their salts, more particularly celecoxib, deracoxib, valdecoxib, parecoxib and its salts, rofecoxib, etoricoxib, lumiracoxib, 4-[5-(4-fluorophenyl)-3-(trifluoromethyl)-1H-pyrazol-1-yl]benzeneulfonamide and 4-[5-(phenyl)-3-(trifluoromethyl)-1H-pyrazol-1-yl]benzenesulfonamide are useful in the method and composition of the invention.

Valdecoxib used in compositions of the invention can be prepared by any known process, for example in the manner set forth in U.S. Pat. No. 5,633,272 to Talley et al. Parecoxib and salts thereof used in compositions of the invention can be prepared by any known process, for example in the manner set forth in U.S. Pat. No. 5,932,598 to Talley et al. Rofecoxib used in compositions of the invention can be prepared by any known process, for example in the manner set forth in U.S. Pat. No. 5,474,995 to Ducharme et al. Etoricoxib used in compositions of the invention can be prepared by any known process, for example in the manner set forth in International Patent Publication No. WO 98/03484. 2-(3,5-Difluorophenyl)-3-[4-(methylsulfonyl) phenyl]-2-cyclopenten-1-one used in compositions of the invention can be prepared by any known process, for example in the manner set forth in European Patent No. 0 863 134. Deracoxib used in compositions of the invention can be prepared by any known process, for example in the manner set forth in U.S. Pat. No. 5,760,068 to Talley et al. 2-(3,4-Difluorophenyl)-4-(3-hydroxy-3-methyl-1-butoxy)-5-[4-(methylsulfonyl)phenyl]-3-(2H)-pyridazinone used in compositions of the invention can be prepared by any known process, for example in the manner set forth in International Patent Publication No. WO 00/24719. Other selective COX-2 inhibitory drugs can be prepared by any known process, including processes set forth in patent publications disclosing such drugs; for example in the case of celecoxib in above-cited U.S. Pat. No. 5,466,823 or in U.S. Pat. No. 5,892,053 to Zhi et al. All patents and publications cited above are incorporated herein by reference.

A preferred concentration range for a selective COX-2 inhibitor in a composition of the invention for intramammary infusion is about 0.01 to about 1000 mg/ml, more preferably about 0.1 to about 750 mg/ml, and still more preferably about 5 to about 250 mg/ml.

A composition of the invention can be admixed with any conventional pharmaceutical additive that does not deleteriously react with other ingredients of the composition. Such additives include, but are not limited to, diluents, antioxidants, preservatives, stabilizers, thickening agents, suspending agents, dispersing agents, solubilization agents, isotonic agents, buffering agents, wetting agents, lubricants, emulsifiers, salts for influencing osmotic pressure, coloring agents, alcohols, other surfactants and conventional pharmaceutical additives and the like, and combinations thereof.

Illustrative excipients include without limitation tocopherols, ascorbyl palmitate, butyl hydroxyanisole, butyl hydroxytoluene, benzoic acid and derivatives thereof, ascorbic acid and salts thereof, e.g., sodium ascorbate, methionine, ethylenediamine, sodium bisulfite, sulfur dioxide, maleic acid, propyl gallate, parabens, chlorobutanol, phenol, sorbic acid and salts thereof, thimerosal, colloidal silica, petrolatum, aluminum stearate, magnesium stearate, talc, sorbitol, dextran, dextrose, lanolin, ceresin, spermaceti, chitosan, paraffin, cellulose ether polymers, starch, propylene glycol, dipropylene glycol, hexylene glycol, polyethylene glycol, ethanol, carrageenan, 12-hydroxystearin, polyvinylpyrrolidone, hydroxyethylpropylcellulose, hydroxyethylcellulose, hydroxypropylmethylcellulose, natural gums such as guar, xanthan and tragacanth gums, silicic acid, carbohydrates, cellosolves such as methyl cellosolve and ethyl cellosolve, vegetable oils and waxes containing at least about 12 carbons in a straight chain, e.g., olive oil and castor oil, trisodium orthophosphate, sodium bicarbonate, N-methylglucamine, L(+)-lysine, L(+)-arginine, acetic acid, boric acid, citric acid, lactic acid, phosphoric acid, hydrochloric acid, sodium hydroxide, sodium phosphate, potassium phosphate, potassium citrate, sodium lactate, mono-, di- and triethanolamines, 2-amino-2-(hydroxymethyl)-1,3-propanediol, tris-hydroxymethylaminomethane, citrate/dextrose, sodium bicarbonate, ammonium chloride, esters such as amyl acetate, ethyl acetate and benzyl benzoate and the like, and combinations thereof.

The vehicle for a liquid composition suitable for intramammary infusion can be aqueous or non-aqueous and is typically composed predominantly of one or more pharmaceutically acceptable diluents, herein referred to as carriers. Examples of non-aqueous carriers include, but are not limited to, vegetable oils, mineral oils, synthetic oils and combinations thereof. Examples of fully saturated non-aqueous carriers include, but are not limited to, esters of medium to long chain fatty acids (such as fatty acid triglycerides with a chain length of about C₆ to about C₂₄). Mixtures of fatty acids are split from the natural oil (for example coconut oil, palm kernel oil, babassu oil or the like) and are refined. In some embodiments, medium chain (about C₈ to about C₁₂) triglycerides are useful. An illustrative saturated non-aqueous carrier comprises capric acid (about 20% to about 45%) and caprylic acid (about 45% to about 80%). Other fully saturated non-aqueous carriers include, but are not limited to, saturated coconut oil (which typically includes a mixture of lauric, myristic, palmitic, capric and caproic acids), including those sold under the Miglyol™ trademark from Huls and bearing trade designations 810, 812, 829 and 840). Also noted are the NeoBee™ products sold by Drew Chemicals. Isopropyl myristate is another example of a non-aqueous carrier useful in compositions of the invention. Examples of synthetic oils include triglycerides and propylene glycol diesters of saturated or unsaturated fatty acids having 6 to 24 carbon atoms, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, heptadecanoic, eicosanoic, heneicosanoic, docosanoic and lignoceric acids and the like. Examples of unsaturated carboxylic acids include oleic, linoleic and linolenic acids and the like. It is understood that the non-aqueous carrier can comprise the mono-, di- and triglyceryl esters of fatty acids or mixed glycerides and/or propylene glycol diesters wherein at least one molecule of glycerol has been esterified with fatty acids of varying carbon atom length. A non-limiting example of a “non-oil” useful as a carrier in compositions of the invention is polyethylene glycol.

Preferred non-aqueous carriers are vegetable oils such as cottonseed oil, corn oil, sesame oil, soybean oil, olive oil, fractionated coconut oil, peanut oil, sunflower oil, safflower oil, almond oil, avocado oil, palm oil, palm kernel oil, babassu oil, beechnut oil, linseed oil, rape oil and the like. The most preferred non-aqueous carrier is cottonseed oil. By way of example cottonseed oil is available in a preparation of 70% unsaturated fatty acids from Sigma Chemical Co.

In one embodiment the carrier has not been modified to contain an increased level of oxidation products, through physical, chemical or mechanical means.

It will be appreciated that preferred amounts of compositions to be administered in a specific case will vary according to the specific composition being utilized, the mode of application, the particular situs and organism being treated, and other factors. Dosages for a given purpose can be determined using conventional considerations, for example, by customary comparison of the differential activities of the subject compositions and of a known agent, e.g., by means of an appropriate conventional pharmaceutical protocol.

An illustrative suspension of the invention containing an antibacterial agent, e.g., ceftiofur hydrochloride, and a selective COX-2 inhibitor, e.g., deracoxib, has the following composition:

| | |
|---|---|
| antibacterial agent | 1-150 mg/ml |
| selective COX-2 inhibitor | 1-350 mg/ml |
| Labrafil ™ M-1944CS | 1-75% |
| microcrystalline wax | 0.1-25% |
| cottonseed oil | q.s. to 100% |

(all percentages are weight/volume).

### EXAMPLES

The following examples illustrate aspects of the present invention but should not be construed as limitations.

### Example 1

An antibacterial suspension to be administered by intramammary infusion is prepared having the following composition:

| | |
|---|---|
| ceftiofur hydrochloride (micronized) | 12.5 mg/ml |
| Labrafil ™ M-1944CS | 200 mg/ml |
| microcrystalline wax NF | 100 mg/ml |
| cottonseed oil NF | q.s. |

The microcrystalline wax and cottonseed oil are heated to 85-98° C. with mixing, in a manufacturing tank. After the microcrystalline wax is completely melted, the mixture is cooled to 38-45° C. and the Labrafil™ M-1944CS is added to the manufacturing tank with mixing to form the vehicle. Ceftiofur hydrochloride is added to the resulting vehicle and mixed to form a uniform suspension. The suspension is screened and filled into 12 ml high density polyethylene mastitis syringes. The packaged product is terminally sterilized by gamma irradiation at a dose of 25-40 kGy.

A selective COX-2 inhibitor suspension to be administered by intramammary infusion is prepared having the following composition:

| | |
|---|---|
| parecoxib free acid | 100 mg/ml |
| Labrafil ™ M-1944CS | 150 mg/ml |
| microcrystalline wax NF | 75 mg/ml |
| cottonseed oil NF | q.s. |

The microcrystalline wax and cottonseed oil are heated to 85-98° C. with mixing, in a manufacturing tank. After the microcrystalline wax is completely melted, the mixture is cooled to 38-45° C. and Labrafil™ M-1944CS is added to the manufacturing tank with mixing to form the vehicle. The parecoxib is added to the resulting vehicle and mixed to form a uniform suspension. The suspension is screened and filled into 12 ml high density polyethylene mastitis syringes. The packaged product is terminally sterilized by gamma irradiation at a dose of 25-40 kGy.

The above suspensions are administered by intramammary infusion to each infected quarter of an udder of a lactating cow at a dose of 125 mg ceftiofur hydrochloride/quarter/day (for 2 to 8 days) and 1,200 mg parecoxib/quarter/day. The suspensions are effective in treatment of lactating cow mastitis.

### Example 2

A suspension to be administered by intramammary infusion is prepared having the following composition:

| | |
|---|---|
| ceftiofur crystalline free acid (micronized) | 25 mg/ml |
| deracoxib | 170 mg/ml |
| Labrafil ™ M-1966CS | 100 mg/ml |
| microcrystalline wax NF | 50 mg/ml |
| corn oil NF | q.s. |

The microcrystalline wax and the corn oil are heated to 85-98° C. with mixing, in a manufacturing tank. After the microcrystalline wax is completely melted, the mixture is cooled to 30-45° C. and the Labrafil™ M-1966CS is added to the manufacturing tank with mixing to form a vehicle. The ceftiofur crystalline free acid and the deracoxib are added to the vehicle and mixed to form a uniform suspension. The suspension is screened and filled into 12 ml high density polyethylene mastitis syringes. The packaged product is terminally sterilized by gamma irradiation at a dose of 25-40 kGy.

The above suspension is administered to all four quarters an udder of a dry cow at a dose of 500 mg ceftiofur crystalline free acid/quarter and 3,400 mg deracoxib/quarter by intramammary infusion. The suspension is effective in treatment of dry cow mastitis.

### Example 3

A suspension to be administered by intramammary infusion is prepared having the following composition:

| | |
|---|---|
| ceftiofur hydrochloride (micronized) | 50 mg/ml |
| deracoxib | 300 mg/ml |
| Labrafil ™ M-1944CS | 50 mg/ml |
| microcrystalline wax NF | 70 mg/ml |
| cottonseed oil NF | q.s. |

The microcrystalline wax and approximately 27% of the total amount of the cottonseed oil are heated to 85-98° C. with mixing, in a kettle. The balance of the cottonseed oil is heated to 85-98° C. with mixing, in a manufacturing tank. After the microcrystalline wax is completely melted, the microcrystalline wax/cottonseed oil mixture in the kettle is transferred to the manufacturing tank containing cottonseed oil and mixed thoroughly. The resulting mixture is cooled to 38-45° C. and the Labrafil™ M-1944CS is added to the manufacturing tank with mixing to form the vehicle. The ceftiofur hydrochloride and deracoxib are added to the resulting vehicle and mixed to form a uniform suspension. The suspension is screened and filled into 12 ml high density polyethylene mastitis syringes. The packaged product is terminally sterilized by gamma irradiation at a dose of 25-40 kGy.

The above suspension is administered to all four quarters of an udder of a dry cow at a dose of 500 mg ceftiofur hydrochloride/quarter and 12,000 mg deracoxib/quarter by intramammary infusion. The suspension is effective in treatment of dry cow mastitis.

### Example 4

A suspension to be administered by intramammary infusion is prepared having the following composition:

| | |
|---|---|
| ceftiofur sodium (micronized) | 25 mg/ml |
| valdecoxib | 1.5 mg/ml |
| Labrafil ™ WL-2609BS | 75 mg/ml |
| microcrystalline wax NF | 100 mg/ml |
| Miglyol ™ 812 | q.s. |

The microcrystalline wax and approximately 30% of the total amount of the Miglyol™ 812 are heated to 85-98° C. with mixing, in a kettle. The balance of the Miglyol™ 812 is heated to 85-98° C. with mixing, in a manufacturing tank. After the microcrystalline wax is completely melted, the microcrystalline wax/Miglyol™ 812 mixture in the kettle is transferred to the manufacturing tank containing the Miglyol™ 812 and mixed thoroughly. The resulting mixture is cooled to 38-45° C. and the Labrafil™ WL-2609BS is added to the manufacturing tank with mixing to form the vehicle. The ceftiofur sodium and the valdecoxib are added to the resulting vehicle and mixed to form a uniform suspension. The suspension is screened and filled into 12 ml high density polyethylene mastitis syringes. The packaged product is terminally sterilized by gamma irradiation at a dose of 25-40 kGy.

The above suspension is administered to all four quarters of an udder of a dry cow at a dose of 500 mg ceftiofur sodium/quarter and 30 mg valdecoxib/quarter by intramammary infusion. The suspension is effective in treatment of dry cow mastitis.

The invention having been described in detail and by reference to the preferred embodiments thereof, it will be apparent that modifications and variations are possible without departing from the scope of the appended claims.

What is claimed is:

1. A pharmaceutical composition comprising therapeutically effective amounts of both an antibacterial agent and a selective COX-2 inhibitor, selected from the group consisting of celecoxib, deracoxib, valdecoxib, parecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl )-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(phenyl)-3-(trifluoromethyl)-1 H-pyrazol-1-yl]benzenesulfonamide and compounds having the formula 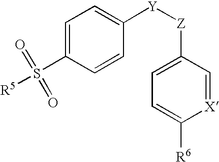
where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, and salts thereof, dispersed in a vehicle comprising (a) a pharmaceutically acceptable non-aqueous carrier, (b) optionally an oil selected from the group consisting of Labrafil™ M-1944CS, Labrafil™ M-1966CS, and Labrafil™ WL-2609BS, and (c) optionally microcrystalline wax wherein said composition, when administered intramammarily, is effective in treatment of an infective condition in an udder of a milk producing animal.

2. The composition of claim 1 wherein the vehicle comprises a pharmaceutically acceptable carrier selected from the group consisting of vegetable oils, mineral oils, medium to long chain fatty acids and alkyl esters thereof, propylene glycol di-esters of medium to long chain fatty acids, mono-, di- and triglyceryl esters of fatty acids, and polyethylene glycols.

3. The composition of claim 1 wherein the vehicle comprises a vegetable oil.

4. The composition of claim 3 wherein the vegetable oil is selected from the group consisting of cottonseed oil, corn oil, sesame oil, soybean oil, olive oil, coconut oil, fractionated coconut oil, peanut oil, sunflower oil, safflower oil, almond oil, avocado oil, palm oil, palm kernel oil, babassu oil, beechnut oil, linseed oil and rape oil.

5. The composition of claim 3 wherein the vegetable oil is cottonseed oil.

6. The composition of claim 36 that further comprises at least one excipient selected from the group consisting of diluents, antioxidants, preservatives, stabilizers, thickening agents, suspending agents, dispersing agents, solubilization agents, isotonic agents, buffering agents, wetting agents, lubricants, emulsifiers, salts for influencing osmotic pressure, coloring agents, alcohols, other surfactants and conventional pharmaceutical additive.

7. A pharmaceutical composition comprising therapeutically effective amounts of both an antibacterial agent and a selective COX-2 inhibitor, selected from the group consisting of celecoxib, deracoxib, valdecoxib, parecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl )-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl )-1H-pyrazol-1-yl]benzenesulfonamide, 4-[5-(phenyl)-3-(trifluoromethyl)-1H-pyrazol-1-yl]benzenesulfonamide, 4-[5-(4-fluorophenyl)-3-(trifluoromethyl)-1H-pyrazol-1-yl]benzenesulfonamide, 4-[5-(phenyl)-3-(trifluoromethyl)-1H-pyrazol-1-yl]benzenesulfonamide and compounds having the formula 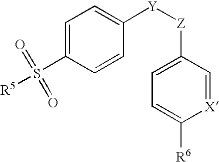
where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, and salts thereof, dispersed in a vehicle comprising (a) a pharmaceutically acceptable non-aqueous carrier, (b) an oil selected from the group consisting of Labrafil™ M-1944CS, Labrafil™ M-1966CS, and Labrafil™ WL-2609BS, and (c) microcrystalline wax wherein said composition, when administered intramammary, is effective in treatment of an infective condition in an udder of a milk producing animal.
